(19) United States
(12) Patent Application Publication
(10) Pub. No.: US 2023/0265064 A1
(43) Pub. Date: Aug. 24, 2023

(54) METHOD FOR PRODUCING A PURIFIED CANNABINOID EXTRACT, AND A PURIFIED CANNABINOID EXTRACT OBTAINED THEREFROM

(71) Applicant: PURCANN PHARMA INC., Québec (CA)

(72) Inventors: Xavier PIGEON, Québec (CA); Claudia CARPENTIER, Québec (CA); Maëlle DERRIEN, Québec (CA); Meryem BOUCHOUCHA, Québec (CA)

(21) Appl. No.: 18/006,463

(22) PCT Filed: Aug. 24, 2021
(86) PCT No.: PCT/CA2021/051169

§ 371 Date: Jan. 23, 2023

Related U.S. Application Data
(60) Provisional application No. 63/073,562, filed on Sep. 2, 2020.

(51) Int. Cl. C07D 311/80; C07C 37/68; C07C 51/48

(52) U.S. Cl. CPC C07D 311/80; C07C 37/685; C07C 51/48

(57) ABSTRACT

The present disclosure relates to a method for producing a purified cannabinoid extract comprising mixing a cannabaceae biomass extract comprising cannabinoid compounds in methanol to obtain a substantially homogeneous methanolic phase; mixing water and the methanolic-phase to obtain a hydro- methanolic-phase; performing a liquid-liquid extraction of the hydro-methanolic phase by using a non-polar solvent whereby cannabinoid compounds are extracted in the non-polar solvent; recovering a non-polar solvent phase comprising the cannabinoid compounds and a rafinate comprising the hydro-methanolic phase; and evaporating the non-polar solvent from the non-polar solvent phase comprising the cannabinoid to provide the purified cannabinoid extract.

### CROSS-REFERENCE TO RELATED APPLICATION

The present application is claiming priority from U.S. Provisional Application No. 63/073,562 filed Sep. 2, 2020, the content of which is hereby incorporated by reference in its entirety.

### FIELD OF THE DISCLOSURE

The present disclosure relates to a method for producing a purified cannabinoid extract.

### BACKGROUND

The cannabinoid molecules have been studied for various reasons over the past decades. In the recent years, the use of cannabinoids as pharmaceutical, cosmetic or recreational products has increased in several countries.

Currently, the major sources of cannabinoids are coming from the extraction of Hemp and/or cannabis biomass. Cannabinoids have varying effects and treatment modalities. Due to the nature of used biomass, cannabinoids are presented together in the extract in different proportions and it is a challenge to separate them from each other. Because of this, a variety of techniques have been developed to extract different cannabinoids and the most common ways to proceed are based on chromatography or distillation methods. These ways have many advantages and disadvantages and there is still a need for innovative methods to purify and refine cannabinoid extracts.

Tetrahydrocannabinolic acid (THCA) or its decarboxylated form (tetrahydrocannabinol (THC)) and cannabidiolic acid (CBDA) or its decarboxylated product (cannabidiol (CBD)) are the most known cannabinoids. For medicinal and cosmeceutical purposes, purified cannabinoids, especially cannabinoids with high CBDA/CBD content and without THCA/THC, are particularly sought after. Indeed, THCA/THC are known to elicit psychoactive effects and are associated with many undesirable side effects. However, CBD/CBDA are non-psychoactive at typical doses, have neuroprotective effects and are known to have anti-inflammatory activity in addition to providing antagonism of THC-induced anxiety.

Thus, there is a need for purified cannabinoid extracts, and methods for producing same.

### SUMMARY OF THE DISCLOSURE

An aspect relates to a method for producing a purified cannabinoid extract, comprising mixing a cannabaceae biomass extract comprising cannabinoid compounds in methanol to obtain a substantially homogeneous methanolic phase; mixing water and said methanolic-phase to obtain a hydro- methanolic- phase; performing a liquid-liquid extraction of said hydro-methanolic phase by using a non-polar solvent whereby cannabinoid compounds are extracted in the non-polar solvent; recovering a non-polar solvent phase comprising said cannabinoid compounds and a rafinate comprising said hydro-methanolic phase; and evaporating non-polar solvent from said non-polar solvent phase comprising said cannabinoid to provide the purified cannabinoid extract.

A further aspect relates to a purified cannabinoid extract prepared by the method as defined herein.

In an embodiment, the cannabaceae biomass extract is Hemp, a cannabis extract or a mixture thereof.

In a further embodiment, the cannabis extract is from Hemp, Cannabis Sativa, Cannabis Indica, a hybrid of Cannabis Sativa and Indica and Cannabis ruderalis.

In another embodiment, the method described herein further comprises the step of pre-treating the cannabaceae biomass prior to mixing in methanol.

In an embodiment, the cannabaceae biomass is pre-treated by drying.

In a further embodiment, the cannabaceae biomass is dried, reducing moisture of the cannabaceae biomass to less than about 15% (wt/wt ratio).

In a further embodiment, the cannabaceae biomass is pre-treated by grinding.

In another embodiment, the cannabaceae biomass is grinded to pieces of about 1 to 0.1 inch long.

In a further embodiment, ein the cannabaceae biomass is grinded to pieces of about 0.2 inch long.

In another embodiment, the non-polar solvent is a straight, branched, or saturated hydrocarbon comprising 5 to 10 carbon atoms.

In a further embodiment, the non-polar solvent is ethanol, or an alkane.

In an embodiment, the alkane is propane, butane, isobutane, pentane, isopentane, neopentane, hexane, 2-methylpentane, 3-methylpentane, 2,2-dimethylbutane, 2,3-dimethylbutane, heptane, methylhexane, dimethylpentane, octane, decane, their isomers or a mixture thereof.

In a further embodiment, the volume ratio of the non-polar solvent to the volume of the hydro-alcoholic phase is ranging from about 0.1-1:1 or about 0.5-1:1.

In another embodiment, the volume ratio of the non-polar solvent to the volume of the hydro-alcoholic phase is about 0.5:1.

In a further embodiment, the cannabinoid extract is purified at room temperature.

In another embodiment, the non-polar solvent is cold ethanol.

In a further embodiment, the total weight amount of cannabinoid compounds is from about 15% to about 60% (wt/wt) relative to the weight of the cannabaceae biomass extract.

IUn an embodiment, the cannabaceae biomass extract crude extract comprises a mixture of two or more cannabinoids compounds selected from THC, cannabichromene (CBC), cannabidiol (CBD), cannabigerol (CBG), cannabinol (CBN), cannabinodiol (CBND), cannabicyclol (CBL), cannabielsoin (CBE), cannabicitran type (CBCT), cannabitriol (CBT), cannabielsoin (CBL), their corresponding carboxylated form, sesquicannabigerol, cannabicoumarononic acid, cannabinoid benzoquinone, and cannabispiroindane.

In a further embodiment, the cannabaceae biomass crude extract comprises CBD, THC, CBDA (cannabidiolic acid) and THCA (tetrahydrocannabinolic acid).

In a further embodiment, the cannabaceae biomass crude extract comprises a ratio CBD/CBDA on THC/THCA between 100 and 5 (wt/wt).

In another embodiment, the purified cannabinoid extract comprises a mass ratio of at least one cannabinoid relative to the total mass of the purified cannabinoid extract that is higher than the corresponding mass ratio of said at least one cannabinoid in said cannabaceae biomass extract.

In a further embodiment, the mass ratio is at least about 1.2.

In another embodiment, the mass ratio is at least about 1.25.

In a further embodiment, the mass ratio is at least about 1.5.

In an embodiment, the mass ratio is at least about 1.8.

In another embodiment, the mass ratio is at least about 2.

In a further embodiment, the method described herein further comprises the step of contacting the cannabaceae biomass extract with activated carbon.

In a further embodiment, the volume ratio of water to methanol in the hydro-methanolic phase is ranging from about 0.5:1 to about 1:0.5.

In an embodiment, the volume ratio of water to methanol in the hydro-methanolic phase is of about 1:1.

In a further embodiment, the method described herein further comprises after the step of recovering said non-polar solvent phase comprising the cannabinoid compounds, performing a second liquid-liquid extraction of the rafinate comprising the hydro-methanolic phase by using a non-polar solvent, recovering a second non-polar solvent phase comprising the cannabinoid compounds and combining the second non-polar solvent phase to the non-polar solvent phase comprising the cannabinoid compounds.

In a further embodiment, the method described herein further comprises a step of washing the non-polar solvent phase or combined non-polar solvent phases with a sodium chloride saturated aqueous solution and recovering a washed non-polar solvent phase.

In a further embodiment, the method described herein further comprises the step of removing water from the non-polar solvent phase and/or the second non-polar solvent phase.

In a further embodiment, the water is removed by contacting a dessicant.

In a further embodiment, the dessicant is MgSO₄, Na SO₄, CaSO₄ or CaCl₂.

In an embodiment, the dessicant is silica.

In a further embodiment, the purified cannabinoid extract comprises decarboxylating carboxylic acid-containing cannabinoids.

In a further embodiment, the total weight amount of cannabinoid compounds relative to the weight of the purified cannabinoid extract is higher than the total weight amount of cannabinoid compounds relative to the weight of said cannabaceae biomass extract.

In an embodiment, the total weight amount of cannabinoid compounds, relative to the weight of the purified cannabinoid extract, is higher by a ratio about 1.25 to about 4 times.

In a further embodiment, the purified cannabinoid extract comprises from about 35 to about 100% (wt/wt) of cannabinoid compounds relative to the weight of said purified cannabinoid extract.

In an embodiment, the purified cannabinoid extract contains about 70% of cannabinoid compounds.

In a further embodiment, the purified cannabinoid extract contains 99% of cannabinoid compounds.

In another embodiment, the total weight amount of CBD and CBDA relative to the weight of the purified cannabinoid extract, is higher than the total weight amount of CBD and CBDA relative to the weight of said cannabaceae biomass extract.

In a further embodiment, the total weight amount of CBD/CBDA, relative to the weight of the purified cannabinoid extract, is higher by a ratio of about 1.5 to about 4 times.

In a further embodiment, the purified cannabinoid extract is comprising from about 30 to 80% (wt/wt) of CBD/CBDA relative to the weight of said purified cannabinoid extract.

In an embodiment, the purified cannabinoid extract is composed from 30% of CBD/CBDA.

In an embodiment, the purified cannabinoid extract is composed from 80% of CBD/CBDA.

### DETAILED DESCRIPTION

The present disclosure relates to a method for producing a purified cannabinoid extract, especially a process to separate and concentrate cannabinoids from other components in a cannabaceae biomass extract.

In one embodiment, the cannabaceae biomass extract is Hemp and/or cannabis extract, including but not limited to Hemp, Cannabis Sativa, Cannabis Indica, a hybrid of Cannabis Sativa and Indica and Cannabis ruderalis. The cannabaceae biomass may be obtained from any part of the cannabaceae plant.

In one embodiment, the used cannabaceae biomass is industrial Hemp.

In one embodiment, the method comprises a further step of pre-treating the cannabaceae biomass.

In one embodiment, said pre-treating step is comprising drying the cannabaceae biomass.

In one embodiment, said pre-treating step is comprising grinding the cannabaceae biomass. The grinding step is intended to cause a size reduction of the biomass. Examples include the shredding of the cannabaceae biomass to pieces about 1 to 0.1-inch-long, preferably about 0.2 inch long.

In one embodiment, said pre-treating step is comprising grinding and drying the cannabaceae biomass.

In one embodiment the drying step is reducing moisture of the cannabaceae biomass to less than about 15% (wt/wt ratio).

In one embodiment, the cannabaceae biomass extract is obtained from cannabaceae biomass extraction process.

In one embodiment, the extraction process for producing a cannabaceae biomass extract is based on the use of supercritical CO₂ technologies. In one embodiment, the extraction process is based on the use of ethanol as solvent for cannabinoids extraction. In one embodiment, the extraction process is based on the use of an alkane as solvent for cannabinoids extraction. The alkane can be propane, butane, isobutane, pentane, isopentane, neopentane, hexane, 2-methylpentane, 3-methylpentane, 2,2-dimethylbutane, 2,3-dimethylbutane, heptane, methylhexane (2 isomers) or dimethylpentane (4 isomers).

In one embodiment, cannabaceae biomass is extracted at room temperature (i.e. from about 20-30° C.). In one embodiment, the Hemp and/or cannabis crude is extracted using cold ethanol. The ethanol temperature can be set from 10° C. to −60° C.

In one embodiment, the extraction solvent is evaporated to produce the cannabaceae biomass extract.

In one embodiment, the total weight amount of cannabinoid compounds is from about 15% to about 60% (wt/wt) relative to the weight of the cannabaceae biomass extract, said extract being solvent evaporation.

In one embodiment, the cannabaceae biomass crude extract contains a mixture of two or more cannabinoid compounds. In the same embodiment, cannabinoid compounds are at least one of THC (such as Δ9-THC, Δ8-THC), cannabichromene (CBC), cannabidiol (CBD), cannabigerol (CBG), cannabinol (CBN), cannabinodiol (CBND), cannabicyclol (CBL), cannabielsoin (CBE), cannabicitran type (CBCT), cannabitriol (CBT), cannabielsoin (CBL), their corresponding carboxylated form or other types (such as sesquicannabigerol; cannabicoumarononic acid; cannabinoid benzoquinone; cannabispiroindane).

In one embodiment, the Hemp and/or cannabis crude extract contains at least CBD and THC and their carboxylated acid form : CBDA (cannabidiolic acid) and THCA (tetrahydrocannabinolic acid) respectively.

In one embodiment, the cannabinoids composition of the Hemp and/or cannabis crude extract is mainly represented by CBD/CBDA and THC/THCA. In one embodiment, the concentration ratio CBD/CBDA on THC/THCA of the Hemp and/or cannabis crude is comprised between 100 and 5 (wt/wt).

The relevant compound structures are summarized in the following table:

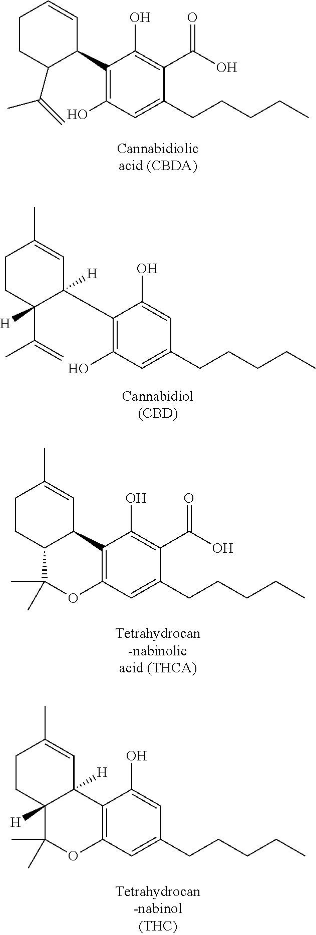

In one embodiment, the purified cannabinoid extract is comprising a mass ratio of at least one cannabinoid, preferably CBD, relative to the total mass of the purified cannabinoid extract, that is higher than the corresponding mass ratio of said at least one cannabinoid, preferably CBD, in said cannabaceae biomass extract. In one embodiment, the ratio is at least about 1.2, or at least about 1.25, or at least about 1.5, or at least about 1.8, or at least about 2.

In one embodiment, the cannabaceae biomass extract can optionally be pre-treated before the purification process. In one embodiment, this pre-treating step is comprising contacting the extract with activated carbon and thereafter removing the used activated carbon. For example, the cannabaceae biomass extract may be eluted through activated carbon.

In one embodiment, the volume ratio of water to methanol in the hydro-methanolic phase is ranging from about 0.5:1 to about 1:0.5, or preferably about 1:1.

The pH of the hydro-methanolic solution does not require to be made more “acidic” or “basic” by the addition of a supplemental acid or base. In other words, the pH is about neutral (a pH of about 7), or may be from 6.5 to 7.5.

The skilled person knows the liquid-liquid extraction technique. The process can be performed using a variety of apparatus, including separatory funnels, and requires a vigorous shaking to mix two immiscible solvents, allowing a transfer of compounds in and out of the phases, followed by a (more or less rapid) separation back into two phases after shaking has ended. The two immiscible phases used herein are a non-polar (organic) phase and a hydromethanolic phase, whereby cannabinoid compounds are extracted in the non-polar solvent.

The non-polar solvent for use herein is subtantially water immiscible. In one embodiment, the non-polar solvent is a straight or branched saturated hydrocarbon comprising 5 to 10 carbon atoms, said hydrocarbon being in liquid state between a temperature of −20° C. and 25° C. In one embodiment, the non-polar solvent is pentane, hexane, heptane, octane, decane or their isomers or a mixture thereof. In one embodiment, the non-polar solvent is hexane.

In one embodiment, the volume ratio of the non-polar solvent to the volume of the hydro-alcoholic phase is ranging from about 0.1-1:1 or about 0.5-1:1 or preferably about 0.5:1.

In one embodiment, after the step of recovering said non-polar solvent phase comprising said cannabinoid compounds, said process is further comprising repeating 1 or more times the steps of performing a further liquid-liquid extraction of said rafinate comprising said hydro-methanolic phase by using a non-polar solvent, recovering a further non-polar solvent phase comprising said cannabinoid compounds and combining said non-polar solvent phases comprising said cannabinoid compounds.

The non-polar solvent and said further non-polar solvent may be the same or different.

In one embodiment, after the step of recovering said non-polar solvent phase comprising said cannabinoid compounds or combining said non-polar solvent phases comprising said cannabinoid compounds, the process is further comprising a step of washing the non-polar solvent phase or combined non-polar solvent phases with a sodium chloride saturated aqueous solution and recovering a washed non-polar solvent phase.

In one embodiment, after the step of recovering said non-polar solvent phase comprising said cannabinoid compounds or the step of combining said non-polar solvent phases comprising said cannabinoid compounds, or after the step of recovering the washed non-polar solvent phase, the process is further comprising a step of substantially removing water from the non-polar solvent phase(s).

In one embodiment, the step of removing water from the non-polar solvent phase(s) is comprising contacting a dessicant with said non-polar solvent phase(s) and removing said dessicant to provide a substantially water-free non-polar solvent phase(s). In one embodiment, the dessicant is an inorganic salt. In one embodiment, the dessicant is MgSO₄, Na₂SO₄, CaSO₄ or CaCl₂. In one embodiment, the dessicant is silica.

The step of evaporating the non-polar solvent to provide the purified cannabinoid extract is known in the art and may be performed by using conventional evaporators, preferably at reduced pressure, while heating or not. The evaporation step is also suceptible to removing any “volatile” compound under these conditions. The resulting purified cannabinoid extract is therefore substantially free of solvent, in particular any remaining non-polar solvent or residual methanol from the liquid-liquid extraction of the hydro-methanolic.

In one embodiment, the purified cannabinoid extract is further comprising decarboxylating carboxylic acid-containing cannabinoids.

In one embodiment, the total weight amount of cannabinoid compounds relative to the weight of the purified cannabinoid extract is higher than the total weight amount of cannabinoid compounds relative to the weight of said cannabaceae biomass extract.

In one embodiment, the total weight amount of cannabinoid compounds, relative to the weight of the purified cannabinoid extract, is higher by a ratio of about 1.25 to about 4 times.

In one embodiment, the purified cannabinoid extract is comprising from about 35 to about 100% (wt/wt) of cannabinoid compounds relative to the weight of said purified cannabinoid extract. In one embodiment, the purified cannabinoid extract contains about 70% of cannabinoid compounds. In one embodiment, the purified cannabinoid extract contains 99% of cannabinoid compounds.

In one embodiment, the total weight amount of CBD and CBDA, relative to the weight of the purified cannabinoid extract, is higher than the total weight amount of CBD and CBDA relative to the weight of said cannabaceae biomass extract.

In one embodiment, the total weight amount of CBD/CBDA, relative to the weight of the purified cannabinoid extract, is higher by a ratio of about 1.5 to about 4 times.

In one embodiment, the purified cannabinoid extract is comprising from about 30 to 80% (wt/wt) of CBD/CBDA relative to the weight of said purified cannabinoid extract. In one embodiment, the purified cannabinoid extract is composed from 30% of CBD/CBDA. In one embodiment, the purified cannabinoid extract is composed from 80% of CBD/CBDA.

### EXAMPLES

The following reagents, equipment and conditions were used.

Ethanol 190 proof, food grade was obtained from Greenfield. MEOH 100% HPLC grade, hexane, acetonitrile, ammonium formate and formic acid were obtained from Sigma. Cannabinoid standards (THC, THCA, CBN, CBD, CBDA) were obtained from Cerrilant.

The cannabinoid biomass/plants were stored in polyethylene Zyploc®-type bags.

Flash drying consisted of a biomass drying at high temperature (i.e. 600-800° C.) during a short period of time (5-10 seconds) to produce biomass having 10-20% humidity.

All chromatographic runs were carried out using Agilent 1100 HPLC Series, consisting of a G1322A solvent degasser, a G1311A quaternary solvent pump, a G1313A autosampler, a G1316A column compartment and a G1315B photodiode-array detector. For this study, the detector was set to 230 nm. Chromatographic separations were performed using a SiliaChrom dt C18 3 μm 4.6×150mm. Column temperature was set to 35° C. and the flow was set to 1.5 mL/min. The analytical method was based on De Backer et al. (Innovative Development and Validation of an HPLC/DAD Method for the Qualitative and Quantitative Determination of Major Cannabinoids in Cannabis Plant Material. J. chromatogr. B (2009), 877, pp 4115-4124), the content of which is incorporated herein by reference. In summary, samples were prepared by dilution in 100% HPLC grade MeOH, to a concentration expected to be near 50 ppm (i.e. center of the standard calibration curve). The injection volume was 10 μL and the sample was eluted under isocratic condition with 75/25 (v/v) acetonitrile/ammonium formate 50 mM +0.1% (v/v) formic acid.

### Preparation of Biomass Crude Extract

The general process can be described as follows: the whole hemp plants, cultivar CANMA, were collected and stored in bags after flash drying. Extraction of non-grinded plant biomass (size of biomass chunk comprised between 2 cm and 0.1 mm) was done in ethanol, at various temperatures, followed by filtration and evaporation of the volatile portion.

### Example 1

### Enriched Extract with Cannabinoids

In this example, 320 g of hemp was extracted using 3800 mL of ethanol at room temperature for 15 minutes, and the insoluble material was filtered off. An additional amount of 320 g of hemp was added to the ethanolic filtrate, followed by a further extraction for 15 minutes followed by filtration. The process was repeated a third time and the extraction solvent was then evaporated to provide the Hemp crude extract.

1.59 g of Hemp crude extract (i.e. Hemp crude oil) was diluted in 20 mL methanol and then 20 mL of water was added. The cannabinoid extraction was performed by adding 20 mL of hexane to the obtained hydro-alcoholic solution. The mixture was mixed vigourously. After the phases had separated, the organic phase comprising hexane was recovered. Then, a second extraction was performed by adding 20 mL of hexane to the recovered aqueous phase. The mixture was mixed vigourously. After the phases had separated, the organic phase comprising hexane was recovered. All the obtained organic phases were combined together. The combined organic phase was washed with an aqueous brine solution and the organic phase was recovered. 300 mg of activated carbon powder was added to the latter. After 2 h, the mixture was filtered and 200 mg of MgSO₄ was added. Then the mixture was filtered and the volatiles in the organic phase were evaporated. 0.44 g of the purified cannabinoid extract was obtained.

TABLE 1
Summary of data example 1.

| | Initial | | |
|---|---|---|---|
| | concentration | Concentration in the | Concentration in the |
| | in plant | Hemp crude extract | purified cannabinoid |
| | (industrial | (after solvent | extract (after |
| | Hemp) | evaporation) | solvent evaporation) |
| Total CBD (% | 1.06 | 24.63 | 30.09 |
| m/m) | | | |
| Total THC (% | <0.3% | 2.01 | 2.8 |
| m/m) | | | |

### Example 2

### High Cannabidiol Content Extract

In this example, the Hemp crude extract was prepared as described in Example 1, except that the extraction in ethanol was conducted at −20° C. instead of room temperature and the ethanolique hemp extract was eluted through activated carbon filter with a low backpressure and then the extraction solvent was evaporated to provide the Hemp crude extract.

The obtained Hemp crude extract was diluted in 20 mL methanol and then 20 mL of water was added. Cannabinoid extraction was performed by adding 20 mL of hexane to the obtained hydro-alcoholic solution. The mixture was mixed vigourously. After the phases had separated, the organic phase comprising hexane was recovered. A second extraction was performed by adding 20 mL of hexane to the recovered aqueous phase. The mixture was mixed vigourously. After the phases had separated, the organic phase comprising hexane was recovered. All the obtained organic phases were combined together. The combined organic phase was washed with an aqueous brine solution and the organic phase was recovered. 300 mg of activated carbon powder was added to the latter. After 2 h, the mixture was filtered and 200 mg of MgSO₄ was added. Then the mixture was filtered and the volatiles in the organic phase were evaporated. 0.44 g of the purified cannabinoid extract was then obtained.

TABLE 2
Summary of data example 2.

| | | Concentration in | Concentration in |
|---|---|---|---|
| | Initial | the Hemp | the purified |
| | concentration | crude extract | cannabinoid |
| | in plant | after eluted | extract |
| | (industrial | through activated | (after solvent |
| | Hemp) | carbon filter | evaporation) |
| Total CBD (% m/m) | 1.06 | 43.3 | 76.0 |
| Total THC (% m/m) | <0.3% | 2.01 | 1.94 |

1. A method for producing a purified cannabinoid extract, comprising:
mixing a cannabaceae biomass extract comprising cannabinoid compounds in methanol to obtain a substantially homogeneous methanolic phase;
mixing water and said methanolic-phase to obtain a hydro- methanolic- phase;
performing a liquid-liquid extraction of said hydro-methanolic phase by using a non-polar solvent whereby cannabinoid compounds are extracted in the non-polar solvent;
recovering a non-polar solvent phase comprising said cannabinoid compounds and a rafinate comprising said hydro-methanolic phase; and
evaporating the non-polar solvent from said non-polar solvent phase comprising said cannabinoid to provide the purified cannabinoid extract.

2. The method of claim 1, wherein the cannabaceae biomass extract is Hemp, a cannabis extract or a mixture thereof.

3. The method of claim 2, wherein the cannabis extract is from Hemp, Cannabis Sativa, Cannabis Indica, a hybrid of Cannabis Sativa and Indica and Cannabis ruderalis.

4. The method of claim 1, further comprising the step of pre-treating the cannabaceae biomass prior to mixing in methanol.

5-9. (canceled)

10. The method of claim 1, wherein the non-polar solvent is a straight, branched, or saturated hydrocarbon comprising 5 to 10 carbon atoms.

11-12. (canceled)

13. The method of claim 1, wherein the volume ratio of the non-polar solvent to the volume of the hydro-alcoholic phase is ranging from about 0.1-1:1 or about 0.5-1:1; preferably the volume ratio of the non-polar solvent to the volume of the hydro-alcoholic phase is about 0.5:1.

14. (canceled)

15. The method of claim 1, wherein the cannabinoid extract is purified at room temperature and/or the non-polar solvent is cold ethanol.

16. (canceled)

17. The method of claim 1, wherein the total weight amount of cannabinoid compounds is from about 15% to about 60% (wt/wt) relative to the weight of the cannabaceae biomass extract.

18. The method of claim 1, wherein the cannabaceae biomass extract crude extract comprises a mixture of two or more cannabinoids compounds selected from THC, cannabichromene (CBC), cannabidiol (CBD), cannabigerol (CBG), cannabinol (CBN), cannabinodiol (CBND), cannabicyclol (CBL), cannabielsoin (CBE), cannabicitran type (CBCT), cannabitriol (CBT), cannabielsoin (CBL), their corresponding carboxylated form, sesquicannabigerol, cannabicoumarononic acid, cannabinoid benzoquinone, and cannabispiroindane.

19. The method of claim 1, wherein the cannabaceae biomass crude extract comprises CBD, THC, CBDA (cannabidiolic acid) and THCA (tetrahydrocannabinolic acid).

20. (canceled)

21. The method of claim 1, wherein the purified cannabinoid extract comprises a mass ratio of at least one cannabinoid relative to the total mass of the purified cannabinoid extract that is higher than the corresponding mass ratio of said at least one cannabinoid in said cannabaceae biomass extract.

22. The method of claim 21, wherein the mass ratio is at least about 1.2, at least about 1.5, at least about 1.8, or at least about 2.

23-26. (canceled)

27. The method of claim 1, further comprising the step of contacting the cannabaceae biomass extract with activated carbon.

28. The method of claim 1, wherein the volume ratio of water to methanol in the hydro-methanolic phase is ranging from about 0.5:1 to about 1:0.5.

29. (canceled)

30. The method of claim 1, further comprising after the step of recovering said non-polar solvent phase comprising said cannabinoid compounds, performing a second liquid-liquid extraction of said rafinate comprising said hydro-methanolic phase by using a non-polar solvent, recovering a second non-polar solvent phase comprising said cannabinoid compounds and combining said second non-polar solvent phase to the non-polar solvent phase comprising said cannabinoid compounds.

31-35. (canceled)

36. The method of claim 1, wherein the purified cannabinoid extract comprises decarboxylating carboxylic acid-containing cannabinoids.

37. The method of claim 1, wherein the total weight amount of cannabinoid compounds relative to the weight of the purified cannabinoid extract is higher than the total weight amount of cannabinoid compounds relative to the weight of said cannabaceae biomass extract.

38. (canceled)

39. The method of claim 1, wherein the purified cannabinoid extract comprises from about 35 to about 100% (wt/wt) of cannabinoid compounds relative to the weight of said purified cannabinoid extract.

40-41. (canceled)

42. The method of claim 18, wherein the total weight amount of CBD and CBDA relative to the weight of the purified cannabinoid extract, is higher than the total weight amount of CBD and CBDA relative to the weight of said cannabaceae biomass extract.

43. (canceled)

44. The method of claim 42, wherein the purified cannabinoid extract is comprising from about 30 to 80% (wt/wt) of CBD/CBDA relative to the weight of said purified cannabinoid extract; wherein the purified cannabinoid extract is composed from 30% of CBD/CBDA; or wherein the purified cannabinoid extract is composed from 80% of CBD/CBDA.

45-47. (canceled)
